(19) United States
(12) Patent Application Publication
(10) Pub. No.: US 2024/0189027 A1
(43) Pub. Date: Jun. 13, 2024

(54) ABLATION SYSTEMS AND METHODS OF USE

(71) Applicant: INTUITIVE SURGICAL OPERATIONS, INC., Sunnyvale, CA (US)

(72) Inventors: Serena Wong, Los Altos, CA (US); Samuel Raybin, Santa Clara, CA (US)

(21) Appl. No.: 18/536,539

(22) Filed: Dec. 12, 2023

Related U.S. Application Data
(60) Provisional application No. 63/387,123, filed on Dec. 13, 2022.

(51) Int. Cl. A61B 18/18

(52) U.S. Cl. CPC A61B 18/1815; A61B 2018/00577

(57) ABSTRACT

An energy delivery system and methods for an ablation procedure may include a generator configured to generate an electrical signal having an operating frequency for an ablation operation and an ablation probe coupled to the generator and configured to receive the electrical signal from the generator. The ablation probe may include a flexible body portion including an inner conductor, an outer conductor, and a dielectric between the inner and outer conductors. The ablation probe may further include a radiating portion electrically coupled to the inner conductor, where the radiating portion is configured to radiate energy received from the electrical signal. A length of the radiating portion may be between 0.35 and 0.65 times an operating wavelength, where the operating wavelength is dependent on the operating frequency of the electrical signal.

### CROSS-REFERENCE TO RELATED APPLICATION

This application claims the priority benefit of U.S. Provisional Application No. 63/387,123, filed Dec. 13, 2022, which is hereby incorporated by reference herein in its entirety.

### FIELD

Disclosed embodiments relate to systems for ablation and methods of use.

### BACKGROUND

Minimally invasive medical techniques are intended to reduce the amount of tissue that is damaged during medical procedures, thereby reducing patient recovery time, discomfort, and harmful side effects. Such minimally invasive techniques may be performed through natural orifices in a patient anatomy or through one or more surgical incisions. Through these natural orifices or incisions, physicians may insert minimally invasive medical instruments (including surgical, diagnostic, therapeutic, and/or biopsy instruments) to reach a target tissue location. Minimally invasive medical tools include instruments such as therapeutic, diagnostic, biopsy, and surgical instruments. Minimally invasive medical tools may also include ablation instruments. Ablation instruments transmit energy in the form of electromagnetic waves to a targeted area of tissue, such as a tumor or other growth, within the patient anatomy to treat (e.g., destroy) the targeted tissue. Some minimally invasive medical tools and ablation instruments may be teleoperated or otherwise computer-assisted. Various features may improve the effectiveness of minimally invasive ablation instruments.

### SUMMARY

The following presents a simplified summary of various examples described herein and is not intended to identify key or critical elements or to delineate the scope of the claims.

In some examples, an energy delivery system may include a generator configured to generate an electrical signal having an operating frequency for an ablation operation and an ablation probe coupled to the generator and configured to receive the electrical signal from the generator. The ablation probe may include a flexible body portion including an inner conductor, an outer conductor, and a dielectric between the inner and outer conductors. The ablation probe may further include a radiating portion electrically coupled to the inner conductor, where the radiating portion is configured to radiate energy received from the electrical signal. A length of the radiating portion is between 0.35 and 0.65 times an operating wavelength being dependent on the operating frequency of the electrical signal.

In some examples, an energy delivery system may include an ablation probe coupled to a generator and having a flexible body portion with an inner conductor and a radiating portion electrically coupled to the inner conductor. A method of operating the energy delivery system may include navigating the ablation probe to position the radiating portion at a target location within a patient, generating an electrical signal with the generator having an operating frequency, and radiating energy received from the electrical signal with the radiating portion to ablate tissue of the patient at the target location and provide a reflectivity to deliver a significant portion of the energy to the tissue, where a length of the radiating portion is between 0.35 and 0.65 times an operating wavelength being dependent on the operating frequency of the electrical signal.

It is to be understood that both the foregoing general description and the following detailed description are illustrative and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTIONS OF THE DRAWINGS

FIG. 1 is a simplified diagram of an energy delivery system according to some embodiments.

FIG. 2 is a simplified diagram of an energy delivery system according to some embodiments.

FIG. 3 is a flowchart illustrating a method for transferring energy to an ablation site according to some embodiments.

FIG. 4 is a simplified diagram of a medical system according to some embodiments.

FIG. 5A is a simplified diagram of a medical instrument system according to some embodiments.

FIG. 5B is a simplified diagram of a medical instrument including a medical tool within an elongate device according to some embodiments.

FIGS. 6A and 6B are simplified diagrams of side views of a patient coordinate space including a medical instrument mounted on an insertion assembly according to some embodiments.

Embodiments of the present disclosure and their advantages are best understood by referring to the detailed description that follows. It should be appreciated that like reference numerals are used to identify like elements illustrated in one or more of the figures, wherein showings therein are for purposes of illustrating embodiments of the present disclosure and not for purposes of limiting the same.

### DETAILED DESCRIPTION

In the following description, specific details are set forth describing some embodiments consistent with the present disclosure. Numerous specific details are set forth in order to provide a thorough understanding of the embodiments. It will be apparent, however, to one skilled in the art that some embodiments may be practiced without some or all of these specific details. The specific embodiments disclosed herein are meant to be illustrative but not limiting. One skilled in the art may realize other elements that, although not specifically described here, are within the scope and the spirit of this disclosure. In addition, to avoid unnecessary repetition, one or more features shown and described in association with one embodiment may be incorporated into other embodiments unless specifically described otherwise or if the one or more features would make an embodiment non-functional.

In some instances, well known methods, procedures, components, and circuits have not been described in detail so as not to unnecessarily obscure aspects of the embodiments.

This disclosure describes various instruments and portions of instruments in terms of their state in three-dimensional space. As used herein, the term “position” refers to the location of an object or a portion of an object in a three-dimensional space (e.g., three degrees of translational freedom along Cartesian x-, y-, and z-coordinates). As used herein, the term “orientation” refers to the rotational placement of an object or a portion of an object (e.g., one or more degrees of rotational freedom such as, roll, pitch, and yaw). As used herein, the term “pose” refers to the position of an object or a portion of an object in at least one degree of translational freedom and to the orientation of that object or portion of the object in at least one degree of rotational freedom (e.g., up to six total degrees of freedom). As used herein, the term “shape” refers to a set of poses, positions, and/or orientations measured along an object. As used herein, the term “distal” refers to a position that is closer to a procedural site and the term “proximal” refers to a position that is further from the procedural site. Accordingly, the distal portion or distal end of an instrument is closer to a procedural site than a proximal portion or proximal end of the instrument when the instrument is being used as designed to perform a procedure.

The disclosure relates to an energy delivery system for tissue ablation that includes a generator and an ablation probe including a flexible body portion and a radiating portion. The radiating portion is configured for radiating energy received from an electrical signal from the generator having an operating frequency. The radiating portion may be non-resonant, or not necessarily resonant, at the operating frequency. Additionally, the radiating portion is configured to provide a reflectivity such that a significant portion (e.g., greater than or equal to 90%) of the energy is delivered to tissue. In some examples, the radiating portion may have a length that is between 0.35 and 0.65 times the operating wavelength of the electrical signal, where the operating wavelength is determined by the operating frequency and the speed of the radiated wave in the medium (e.g., tissue) of interest for the ablation procedure. In some examples, the radiating portion can have a length is that generally half of the operating wavelength.

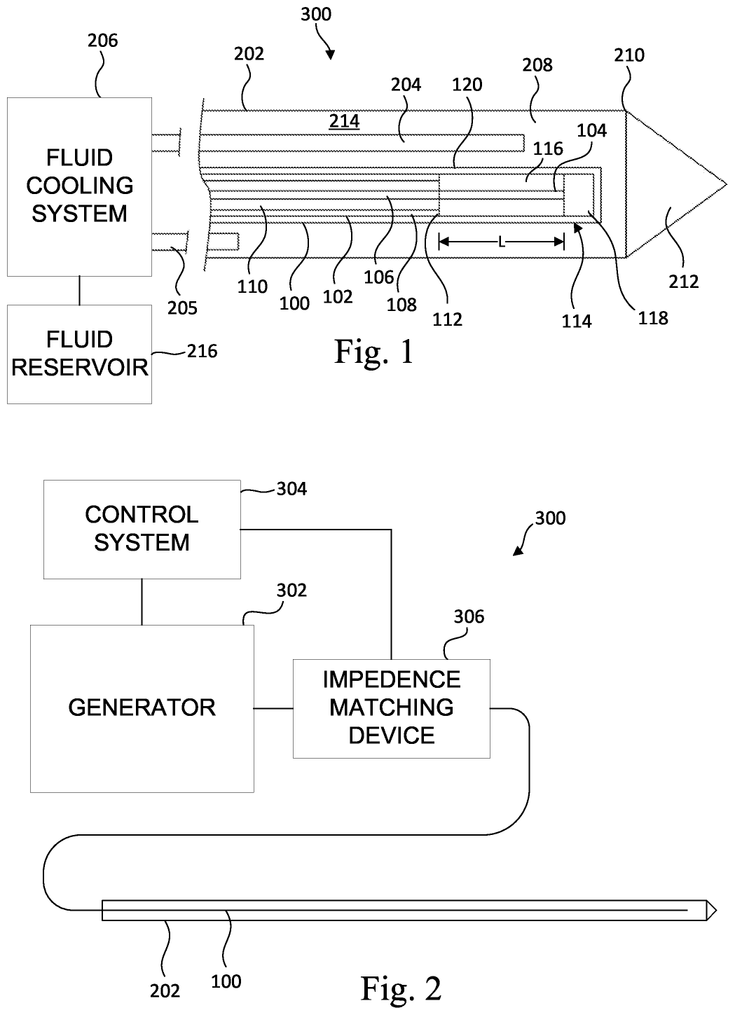

Conventionally, quarter wavelength antennas or antennas with resonance at the frequency of interest have been used for optimum transmission into target tissue. As described herein, it is suggested to operate an ablation probe off resonance, provided that the reflectivity (S11) at the operating frequency remains low enough to be tolerated and deliverable by the generator, e.g. −10 dB and lower. It has been thought previously that half wavelength antennas are not usable because they are off resonance and constitute a higher impedance, reflected power, and low transmission. However, under the build-up of device layers given in the design shown in FIGS. 1 and 2 with the antenna surrounded by a lossy high dielectric material, the S11 can be low enough that a half wavelength design can be tolerated. Furthermore, reflected power can be reduced or eliminated by use of an impedance matching device electrically coupled between a generator and an ablation probe. Furthermore, the energy delivery systems discussed herein advantageously produces desirable circulation ablation patterns in target tissue.

FIGS. 1 and 2 are simplified diagrams of energy delivery systems according to some embodiments. In some embodiments, the energy delivery systems are used for tissue ablation, causing an increase in a temperature of an anatomic target area by transmitting electromagnetic waves from a radiating portion of an ablation probe to the anatomic target area, or ablation site. In some embodiments, ablation probes may be flexible and suitable for use in, for example, surgical, diagnostic, therapeutic, ablative, and/or biopsy procedures. In some embodiments, the energy delivery systems may be used pursuant to an image-guided medical procedure performed with a teleoperated medical system as described in further detail below. While some embodiments are provided herein with respect to such procedures, any reference to medical or surgical instruments and medical or surgical methods is non-limiting. In some embodiments, the energy delivery systems may be used for non-teleoperational procedures involving traditional manually operated medical instruments. The systems, instruments, and methods described herein may be used for animals, human cadavers, animal cadavers, portions of human or animal anatomy, non-surgical diagnosis, as well as for industrial systems and general robotic, general teleoperational, or robotic medical systems.

FIG. 1 is a simplified side cross-sectional view of an ablation probe 100 within an energy delivery system 300 for tissue ablation configured in accordance with some embodiments. As shown in FIG. 1, the ablation probe 100 includes a flexible body portion 102 and a radiating portion 104. The body portion 102 includes an inner conductor 106, an outer conductor 108, and at least one dielectric layer 110 between the inner conductor 106 and the outer conductor 108. The body portion 102 can be elongate having a proximal end (not shown) and a distal portion 112. The radiating portion 104 is coupled to the distal portion 112 of the body portion 102. For example, the radiating portion 104 is electrically coupled to the inner conductor 106 of the body portion 102 to receive energy therefrom. As shown, the radiating portion 104 projects distally away from the body portion 102. The radiating portion 104 may be a portion of the inner conductor 106 (e.g., that is not surrounded by the dielectric layer 110 and the outer conductor 108) or may be a separate component that is attached to the inner conductor 106.

The dielectric layer 110 can at least partially surround the inner conductor 106 to insulate and electrically separate the inner conductor 106 from the outer conductor 108. The inner conductor 106 and outer conductor 108 can each be composed of a conductive material (e.g., copper, aluminum, nitinol, etc.). In some embodiments, the inner conductor 106 and outer conductor 108 are composed of the same conductive material, while in other embodiments the inner conductor 106 and outer conductor 108 can be composed of different conductive materials.

In some embodiments, the inner conductor 106 can be an elongated flexible structure (e.g., one or more wires, filaments, fibers, etc.) extending along the length of the body portion 102 and, in some forms, the radiating portion 104. In one example, the inner conductor 106 can be a solid wire.

In some embodiments, the outer conductor 108 includes at least one multi-filament layer having a plurality of filaments (e.g., ribbons, tapes, wires, fibers, etc.) that are braided or woven around the dielectric 110. The filaments can be made of a conductive material (e.g., copper, aluminum, nitinol, etc.). In another example, the outer conductor 108 can include at least one layer in which a single filament of conductive material is wrapped around the dielectric 110 (e.g., a wrapped metal ribbon). In another example, the outer conductor 108 can include at least one layer of a solid material (e.g., a foil layer, sheet, coating, a tube, etc.). In some embodiments, the outer conductor 108 is composed entirely of a single material and/or structure extending the length of the body portion 102. In other embodiments, different portions of the outer conductor 108 can be composed of different materials and/or structures. For example, the outer conductor 108 can include a combination of braided and wrapped materials, such as a first layer or region composed of a braided material, and a second layer or region composed of a wrapped material. As another example, the outer conductor 108 can include an outer braided multi-filament layer and an inner foil layer. In some examples, the foil layer could made of a material provided for shielding (e.g., copper).

The dielectric 110 can be composed of a non-conductive material. For example, the dielectric 110 can be a polymer material (e.g., solid or foam). The dielectric 110 can be a layer of solid material, or can include one or more filaments of non-conductive material that are braided, woven, or wrapped around the inner conductor 106. As described above, the outer conductor 108 can include one or more layers of material around the dielectric 110.

In some embodiments, the body portion 102 and radiating portion 104 are formed from an elongate device, such as a coaxial cable (e.g., a flexible coaxial cable). The body portion 102 and radiating portion 104 can be integrally formed from a single elongate device, e.g. coaxial cable, such that a proximal section of the elongate device forms the body portion 102 and a distal section of the elongate device, e.g., the inner conductor 106, forms the radiating portion 104. In other forms, the body portion 102 and radiating portion 104 can be separate components and the radiating portion 104 can be subsequently electrically coupled to the inner conductor 106 of the body portion 102.

It will be understood that the structures of the inner conductor 106, outer conductor 108, and/or dielectric 110 can be configured to impart flexibility to the body portion 102 and radiating portion 104.

As shown in FIG. 1, the ablation probe 100 can further include a cap structure 114 that encompasses the radiating portion 104. The cap structure 114 may include a tube 116 with a cylindrical shape that receives the radiating portion 104 at least partially therethrough. The tube 116 can be a nonconductive material, such as polyether ether ketone (PEEK), polytetrafluoroethylene (PTFE), or other suitable materials. The tube 116 may have an inner diameter sized to receive the radiating portion 105 therein and an outer diameter generally identical to the combined diameter of the inner conductor 106, outer conductor 108, and dielectric 110.

The cap structure 114 may further include a plug 118 disposed distally of the tube 116 and radiating portion 104 that is configured to seal the tube 116 and/or other components of the radiating portion 104 from the ingress of fluid. The plug 118 can be made from a non-conductive material, such as glue or another adhesive, a conformal coating (e.g., a parylene coating or vapor-deposited coating), or a reflowable material.

The ablation probe 100 may further include a barrier layer 120 that is positioned over at least a portion of the body portion 102 and the radiating portion 104. The barrier layer 120 can prevent the ingress of fluid into the body portion 102, including the inner conductor 106, the outer conductor 108, and the dielectric 110, and/or radiating portion 104. The barrier layer 120 can also be positioned over and seal the tip of the ablation probe 100, including the cap structure 114 thereof. The barrier layer 120 may be formed of a flexible and fluid impermeable material. In some embodiments, the barrier layer 120 is made from a non-conductive material, such as a polymer, e.g., polyethylene terephthalate (PET)), a plastic, a heat-shrink material, other conformal coating (e.g., a parylene coating or vapor-deposited coating), or a combination thereof. The barrier layer 120 may be thin and form fit around the components contained therein, or may maintain a flexible tubular form.

As shown in FIG. 1, the energy delivery system 300 further includes a jacket 202, at least one fluid conduit 204, and a fluid cooling system 206. The jacket 202 may be an elongated hollow structure having a central lumen or channel 208 extending between a distal end portion 210 and a proximal end portion (not shown). The ablation probe 100 is disposed within the central lumen 208.

In some embodiments, the jacket 202 may be formed from a thermoplastic material or other flexible and fluid impermeable materials. In some embodiments, the jacket 202 is closed, sealed, or otherwise restricts fluid from passing into or out of the jacket 202. For example, the jacket 202 can be coupled to and/or sealed by a tip section 212 at the distal end portion 210 thereof. The tip section 212 may allow the probe 100 to more easily puncture anatomic tissue. The tip section 212 may be formed in any shape, including any number of faces forming the tip, at any angle, and/or with any ratio of sizes (e.g., width vs. length) that will optimize tissue penetration. In the illustrated embodiment, for example, the tip section 212 is a conical structure with a triangular cross-sectional shape. In other embodiments, the tip section 212 can have a different shape, such as a domed, hemispherical, or rounded shape. The tip section 212 can be formed using glass molds or other suitable techniques. The tip section 212 may be composed of fluoropolymers, e.g., ethylene tetrafluoroethylene (ETFE), PEEK, other high temperature plastic materials, and/or other suitable materials. In still further embodiments, one or more portions of the tip section 212 may be radiopaque.

The fluid cooling system 206 can be configured to cool the ablation probe 100 by introducing a coolant (e.g., a fluid 214 or another liquid or gaseous cooling agent) into the central lumen 208 (e.g., also referred to herein as a chamber or channel) of the jacket 202. The fluid 214 may be, for example, water or a saline solution. The fluid cooling system 206 can be coupled to the central lumen 208 and/or the jacket 202 to deliver the fluid 214 into the central lumen 208. The fluid cooling system 206 may include a fluid reservoir 216 (shown schematically) and other components such as pumps, valves, refrigeration systems, suction systems, and/or sensors (not shown). In the illustrated embodiment, for example, the fluid cooling system 206 includes or is coupled to at least one fluid conduit 204 that extends through at least a portion of the central lumen 208 within the jacket 202. The fluid conduit 204 can extend along at least a portion of the ablation probe 100, such as along the body portion 102 and/or the radiating portion 104. The fluid 214 may be directed within the central lumen 208 through the fluid conduit 204. Accordingly, a channel may be formed between the ablation probe 100 and the interior of the jacket 202. In some embodiments, the fluid conduit 204 may extend to a position alongside the radiating portion 104 or to at least a distal end portion of the radiating portion 104 to provide recoverable flexibility and support along a maximized length of the ablation probe 100 and an added benefit of delivering coolant to the distal end portion of the radiating portion 104 for increased and/or more uniform cooling thereof.

The fluid cooling system 206 may be an open loop system, a partially open loop system, a closed loop system, or any other suitable type of cooling system. The fluid conduit 204 may be used, for example, to provide inflow of the fluid 214 to the central lumen 208. The fluid 214 can circulate about the radiating portion 104 and/or the body portion 102 within the central lumen 208, and can return in a proximal direction within the central lumen 208 to be purged in a reservoir (not shown) or purged to the environment. In other embodiments, the fluid 214 can exit the jacket 202 via openings or slits in the jacket 202, or return to the fluid cooling system 206 via the central lumen 208 and/or an outflow fluid conduit 205. Alternatively, the fluid conduit 204 can be used to provide return flow of the fluid 214 from the central lumen 208 by discontinuing inlet fluid from the fluid reservoir 216, reversing flow, and providing suction to the fluid conduit 204 using the fluid cooling system 206. In other embodiments, a separate fluid conduit (not shown) can be provided that does not provide inflow of fluid 214 and is only used for return flow. In some embodiments, the return flow can be purged in a combination of flow through the central lumen 208 in a proximal direction, flow through fluid conduits, and/or through openings in the jacket 202.

As shown in simplified diagram of FIG. 2, the energy delivery system 300 further includes a generator 302 that is configured to generate an electrical signal having an operative wavelength for an ablation operation. The ablation probe 100 is electrically coupled to the generator 302 and configured to receive the electrical signal therefrom. Pursuant to this, the body portion 102 is configured to conduct energy from the generator 302 to the radiating portion 104. The radiating portion 104 is used to radiate energy for use in the ablation operation.

The radiating portion 104 is used to radiate energy within a desired wavelength range. In some embodiments, a length L of the radiating portion 104 is sized to be between 0.35 and 0.65 times an operating wavelength, where the operating wavelength is dependent on the operating frequency of the electrical signal generated by the generator 302. For example, the operating wavelength may be determined by the operating frequency and the speed of the radiated wave in the medium of interest (e.g., tissue to be treated). In other examples, the length L of the radiating portion 104 can be between 0.4 and 0.6 times the operating wavelength, between 0.45 and 0.55 times the operating wavelength, or about 0.5 times the operating wavelength. It has been found that these ranges and values advantageously provide a circular ablation zone within the target tissue.

In some examples, the generator 302 can be configured to generate an electrical signal having an operating wavelength corresponding to a frequency of 2.45 GHz. In other examples, the generator 302 can be configured to generate an electrical signal having an operating wavelength corresponding to a frequency at any of the open frequency bands, including 915 MHZ, 5 GHZ, and/or frequency bands therebetween, for example.

In some embodiments, the energy delivery system 300 may further include a control system 304 operably coupled to the generator 302 and configured to adjust the operating frequency thereof based on a medium/tissue of interest for the ablation procedure. The control system 304 may include processing circuitry that implements the some or all of the methods or functionality discussed herein. The control system 304 may include at least one memory and at least one processor for controlling the operations of the energy delivery system 300. The control system 304 may include instructions (e.g., a non-transitory machine-readable medium storing the instructions) that when executed by the at least one processor, configures the one or more processors to implement some or all of the methods or functionality discussed herein. In some examples, the control system 304 may include other types of processing circuitry, such as application-specific integrated circuits (ASICs) and/or field-programmable gate array (FPGAs). The control system 304 may be implemented using hardware, firmware, software, or a combination thereof.

Ablation probes having a length relative to operating frequency/wavelength as described herein can maintain low reflected power, with return loss (S11) at operating frequencies of interest (e.g., 2.45 GHZ) being such that a significant portion of the energy is delivered to tissue during the ablation procedure. For example, significant portion can correspond to an S11 being below −10 dB during an ablation procedure. In some examples, the S11 corresponds with reflectivity less than 10%. In some embodiments, In some examples, the S11 corresponds with reflectivity less than 5%, or less than 1%. In some embodiments, such as if reflected power levels are too high or it is desirable to otherwise protect the generator 302 or other components of the energy delivery system 300, the system 300 can further include an impedance matching device 306 that is electrically coupled between the generator 302 and the ablation probe 100. The impedance matching device 306 can be utilized to adjust or reduce reflected power seen by the generator 302 by matching the effective impedance to the output impedance of the generator 302 to thereby reduce heating in the output stages of the generator 302. In one example, the impedance matching device 306 can be a mechanical tuner, such as a slide tuner, or various matching networks, such as L, T, and pi networks. The control system 304 may be operably coupled to the impedance matching device 306 to control the operation thereof.

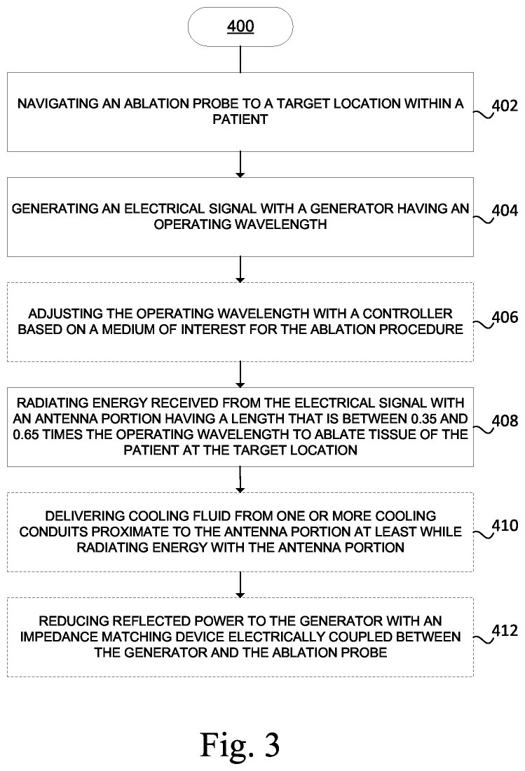

FIG. 3 illustrates a method 400 for transferring energy to an ablation target site according to some embodiments. The method 400 is illustrated as a set of operations or processes 402 through 412. Not all of the illustrated processes may be performed in all embodiments of method 400. Additionally, one or more processes that are not expressly illustrated in FIG. 3 may be included before, after, in between, or as part of the processes 402 through 412. Processes may also be performed in different orders. In some embodiments, one or more of the processes 402 through 412 may be implemented, at least in part, in the form of executable code stored on non-transitory, tangible, machine-readable media that when run by one or more processors (e.g., the processors of a controller) may cause the one or more processors to perform one or more of the processes. In one or more embodiments, the processes 402 through 412 may be performed by a controller (e.g., control system 304).

At process 402, an ablation probe (e.g., ablation probe 100) is navigated to a target location within a patient. The ablation probe can be configured as described herein. In one example, a flexible elongate device navigates the ablation probe to the target location. The ablation probe may be inserted within a lumen of the flexible elongate device. At process 404, an electrical signal is generated with a generator (e.g., generator 302), where the electrical signal has an operating frequency. An operating wavelength corresponding to the operating frequency can be configured as described herein. At optional process 406, a controller (e.g., control system 304) can adjust the operating wavelength based on a medium of interest (and how the properties of the medium would change as a function of temperature, treatment, etc.) or actual measured impedance in the medium for the ablation procedure. The operating frequency may be set or adjusted based on the length of the radiating portion of the ablation probe and the properties of the medium that it is in. At process 408, energy received from the electrical signal is radiated with a radiating portion (e.g., radiating portion 104) of the ablation probe, where the radiating portion has a length that is between 0.35 and 0.65 times the operating wavelength of the electrical signal to ablate tissue at the target location. At process 410, cooling fluid is delivered from one or more cooling conduits (e.g., fluid conduit(s) 204) proximate to the radiating portion at least while the radiating portion is radiating energy. In some examples, cooling is not used while the radiating portion is radiating energy. At optional process 412, reflected power to the generator may be reduced with an impedance matching device (e.g., impedance matching device 306) that is electrically coupled between the generator and the ablation probe. It will be understood that one or more the processes can occur simultaneously.

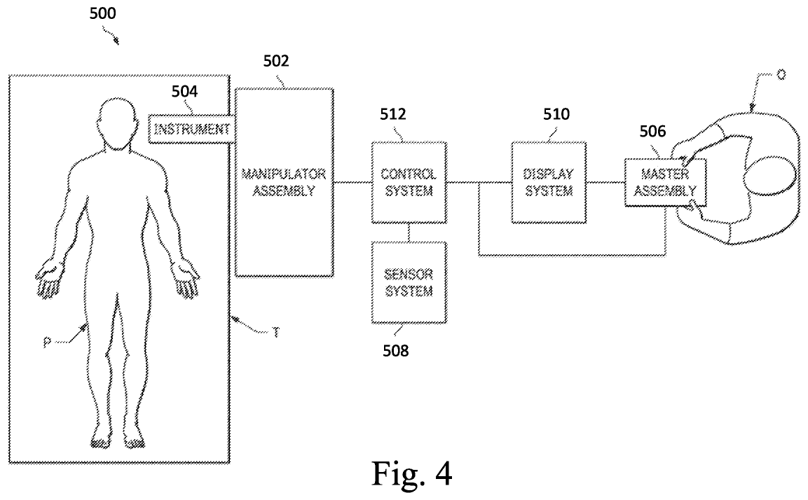

In various embodiments, any of the described energy delivery systems may be used as a medical instrument delivered by, coupled to, and/or controlled by a teleoperated medical system. FIG. 4 is a simplified diagram of a teleoperated medical system 500 according to some embodiments. In some embodiments, teleoperated medical system 500 may be suitable for use in, for example, surgical, diagnostic, therapeutic, or biopsy procedures. While some embodiments are provided herein with respect to such procedures, any reference to medical or surgical instruments and medical or surgical methods is non-limiting. The systems, instruments, and methods described herein may be used for animals, human cadavers, animal cadavers, portions of human or animal anatomy, non-surgical diagnosis, as well as for industrial systems and general robotic or teleoperational systems.

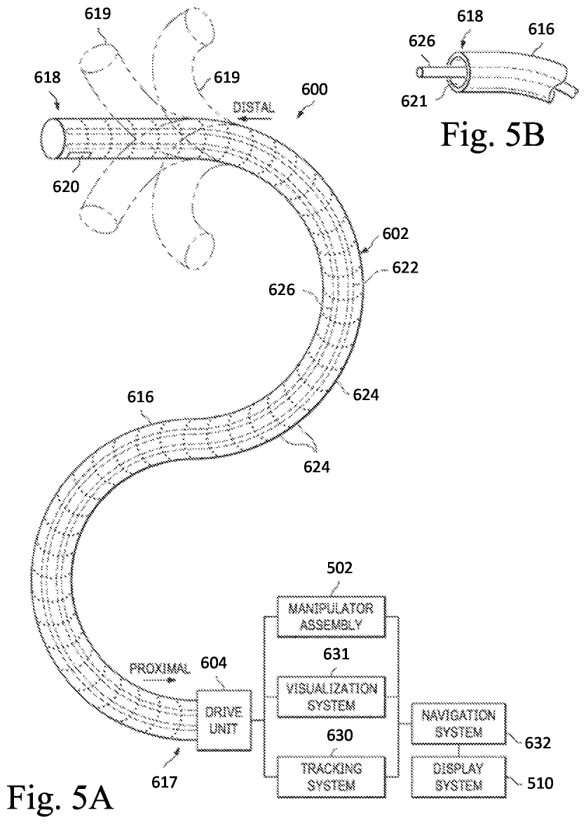

FIG. 5 is a simplified diagram of a medical system 500 according to some embodiments. The medical system 500 may be suitable for use in, for example, surgical, diagnostic (e.g., biopsy), or therapeutic (e.g., ablation, electroporation, etc.) procedures. While some embodiments are provided herein with respect to such procedures, any reference to medical or surgical instruments and medical or surgical methods is non-limiting. The systems, instruments, and methods described herein may be used for animals, human cadavers, animal cadavers, portions of human or animal anatomy, non-surgical diagnosis, as well as for industrial systems, general or special purpose robotic systems, general or special purpose teleoperational systems, or robotic medical systems.

As shown in FIG. 5, medical system 500 may include a manipulator assembly 502 that controls the operation of a medical instrument 504 in performing various procedures on a patient P. Medical instrument 504 may extend into an internal site within the body of patient P via an opening in the body of patient P. The manipulator assembly 502 may be teleoperated, non-teleoperated, or a hybrid teleoperated and non-teleoperated assembly with one or more degrees of freedom of motion that may be motorized and/or one or more degrees of freedom of motion that may be non-motorized (e.g., manually operated). The manipulator assembly 502 may be mounted to and/or positioned near a patient table T. A master assembly 506 allows an operator O (e.g., a surgeon, a clinician, a physician, or other user) to control the manipulator assembly 502. In some examples, the master assembly 506 allows the operator O to view the procedural site or other graphical or informational displays. In some examples, the manipulator assembly 502 may be excluded from the medical system 500 and the instrument 504 may be controlled directly by the operator O. In some examples, the manipulator assembly 502 may be manually controlled by the operator O. Direct operator control may include various handles and operator interfaces for hand-held operation of the instrument 504.

The master assembly 506 may be located at a surgeon's console which is in proximity to (e.g., in the same room as) a patient table T on which patient P is located, such as at the side of the patient table T. In some examples, the master assembly 506 is remote from the patient table T, such as in in a different room or a different building from the patient table T. The master assembly 506 may include one or more control devices for controlling the manipulator assembly 502. The control devices may include any number of a variety of input devices, such as joysticks, trackballs, scroll wheels, directional pads, buttons, data gloves, trigger-guns, hand-operated controllers, voice recognition devices, motion or presence sensors, and/or the like.

The manipulator assembly 502 supports the medical instrument 504 and may include a kinematic structure of links that provide a set-up structure. The links may include one or more non-servo controlled links (e.g., one or more links that may be manually positioned and locked in place) and/or one or more servo controlled links (e.g., one or more links that may be controlled in response to commands, such as from a control system 512). The manipulator assembly 502 may include a plurality of actuators (e.g., motors) that drive inputs on the medical instrument 504 in response to commands, such as from the control system 512. The actuators may include drive systems that move the medical instrument 504 in various ways when coupled to the medical instrument 504. For example, one or more actuators may advance medical instrument 504 into a naturally or surgically created anatomic orifice. Actuators may control articulation of the medical instrument 504, such as by moving the distal end (or any other portion) of medical instrument 504 in multiple degrees of freedom. These degrees of freedom may include three degrees of linear motion (e.g., linear motion along the X, Y, Z Cartesian axes) and in three degrees of rotational motion (e.g., rotation about the X, Y, Z Cartesian axes). One or more actuators may control rotation of the medical instrument about a longitudinal axis. Actuators can also be used to move an articulable end effector of medical instrument 504, such as for grasping tissue in the jaws of a biopsy device and/or the like, or may be used to move or otherwise control tools (e.g., imaging tools, ablation tools, biopsy tools, electroporation tools, etc.) that are inserted within the medical instrument 504.

The medical system 500 may include a sensor system 508 with one or more sub-systems for receiving information about the manipulator assembly 502 and/or the medical instrument 504. Such sub-systems may include a position sensor system (e.g., that uses electromagnetic (EM) sensors or other types of sensors that detect position or location); a shape sensor system for determining the position, orientation, speed, velocity, pose, and/or shape of a distal end and/or of one or more segments along a flexible body of the medical instrument 504; a visualization system (e.g., using a color imaging device, an infrared imaging device, an ultrasound imaging device, an x-ray imaging device, a fluoroscopic imaging device, a computed tomography (CT) imaging device, a magnetic resonance imaging (MRI) imaging device, or some other type of imaging device) for capturing images, such as from the distal end of medical instrument 504 or from some other location; and/or actuator position sensors such as resolvers, encoders, potentiometers, and the like that describe the rotation and/or orientation of the actuators controlling the medical instrument 504.

The medical system 500 may include a display system 510 for displaying an image or representation of the procedural site and the medical instrument 504. Display system 510 and master assembly 506 may be oriented so physician O can control medical instrument 504 and master assembly 506 with the perception of telepresence.

In some embodiments, the medical instrument 504 may include a visualization system, which may include an image capture assembly that records a concurrent or real-time image of a procedural site and provides the image to the operator O through one or more displays of display system 510. The image capture assembly may include various types of imaging devices. The concurrent image may be, for example, a two-dimensional image or a three-dimensional image captured by an endoscope positioned within the anatomical procedural site. In some examples, the visualization system may include endoscopic components that may be integrally or removably coupled to medical instrument 504. Additionally or alternatively, a separate endoscope, attached to a separate manipulator assembly, may be used with medical instrument 504 to image the procedural site. The visualization system may be implemented as hardware, firmware, software or a combination thereof which interact with or are otherwise executed by one or more computer processors, such as of the control system 512.

Display system 510 may also display an image of the procedural site and medical instruments, which may be captured by the visualization system. In some examples, the medical system 500 provides a perception of telepresence to the operator O. For example, images captured by an imaging device at a distal portion of the medical instrument 504 may be presented by the display system 510 to provide the perception of being at the distal portion of the medical instrument 504 to the operator O. The input to the master assembly 506 provided by the operator O may move the distal portion of the medical instrument 504 in a manner that corresponds with the nature of the input (e.g., distal tip turns right when a trackball is rolled to the right) and results in corresponding change to the perspective of the images captured by the imaging device at the distal portion of the medical instrument 504. As such, the perception of telepresence for the operator O is maintained as the medical instrument 504 is moved using the master assembly 506. The operator O can manipulate the medical instrument 504 and hand controls of the master assembly 506 as if viewing the workspace in substantially true presence, simulating the experience of an operator that is physically manipulating the medical instrument 504 from within the patient anatomy.

In some examples, the display system 510 may present virtual images of a procedural site that are created using image data recorded pre-operatively (e.g., prior to the procedure performed by the medical instrument system 600) or intra-operatively (e.g., concurrent with the procedure performed by the medical instrument system 600), such as image data created using computed tomography (CT), magnetic resonance imaging (MRI), positron emission tomography (PET), fluoroscopy, thermography, ultrasound, optical coherence tomography (OCT), thermal imaging, impedance imaging, laser imaging, nanotube X-ray imaging, and/or the like. The virtual images may include two-dimensional, three-dimensional, or higher-dimensional (e.g., including, for example, time based or velocity-based information) images. In some examples, one or more models are created from pre-operative or intra-operative image data sets and the virtual images are generated using the one or more models.

In some examples, for purposes of imaged guided medical procedures, display system 510 may display a virtual image that is generated based on tracking the location of medical instrument 504. For example, the tracked location of the medical instrument 504 may be registered (e.g., dynamically referenced) with the model generated using the pre-operative or intra-operative images, with different portions of the model correspond with different locations of the patient anatomy. As the medical instrument 504 moves through the patient anatomy, the registration is used to determine portions of the model corresponding with the location and/or perspective of the medical instrument 504 and virtual images are generated using the determined portions of the model. This may be done to present the operator O with virtual images of the internal procedural site from viewpoints of medical instrument 504 that correspond with the tracked locations of the medical instrument 504.

The medical system 500 may also include the control system 512, which may include processing circuitry that implements the some or all of the methods or functionality discussed herein. The control system 512 may include at least one memory and at least one processor for controlling the operations of the manipulator assembly 502, the medical instrument 504, the master assembly 506, the sensor system 508, and/or the display system 510. Control system 512 may include instructions (e.g., a non-transitory machine-readable medium storing the instructions) that when executed by the at least one processor, configures the one or more processors to implement some or all of the methods or functionality discussed herein. While the control system 512 is shown as a single block in FIG. 4, the control system 512 may include two or more separate data processing circuits with one portion of the processing being performed at the manipulator assembly 502, another portion of the processing being performed at the master assembly 506, and/or the like. In some examples, the control system 512 may include other types of processing circuitry, such as application-specific integrated circuits (ASICs) and/or field-programmable gate array (FPGAs). The control system 512 may be implemented using hardware, firmware, software, or a combination thereof.

In some examples, the control system 512 may receive feedback from the medical instrument 504, such as force and/or torque feedback. Responsive to the feedback, the control system 512 may transmit signals to the master assembly 506. In some examples, the control system 512 may transmit signals instructing one or more actuators of the manipulator assembly 502 to move the medical instrument 504. In some examples, the control system 512 may transmit informational displays regarding the feedback to the display system 510 for presentation or perform other types of actions based on the feedback.

The control system 512 may include a virtual visualization system to provide navigation assistance to operator O when controlling the medical instrument 504 during an image-guided medical procedure. Virtual navigation using the virtual visualization system may be based upon an acquired pre-operative or intra-operative dataset of anatomic passageways of the patient P. The control system 512 or a separate computing device may convert the recorded images, using programmed instructions alone or in combination with operator inputs, into a model of the patient anatomy. The model may include a segmented two-dimensional or three-dimensional composite representation of a partial or an entire anatomic organ or anatomic region. An image data set may be associated with the composite representation. The virtual visualization system may obtain sensor data from the sensor system 508 that is used to compute an (e.g., approximate) location of the medical instrument 504 with respect to the anatomy of patient P. The sensor system 508 may be used to register and display the medical instrument 504 together with the pre-operatively or intra-operatively recorded images. For example, PCT Publication WO 2016/191298 (published Dec. 1, 2016 and titled “Systems and Methods of Registration for Image Guided Surgery”), which is incorporated by reference herein in its entirety, discloses example systems.

During a virtual navigation procedure, the sensor system 508 may be used to compute the (e.g., approximate) location of the medical instrument 504 with respect to the anatomy of patient P. The location can be used to produce both macro-level (e.g., external) tracking images of the anatomy of patient P and virtual internal images of the anatomy of patient P. The system may include one or more electromagnetic (EM) sensors, fiber optic sensors, and/or other sensors to register and display a medical instrument together with pre-operatively recorded medical images. For example, U.S. Pat. No. 8,900,131 (filed May 13, 2011 and titled “Medical System Providing Dynamic Registration of a Model of an Anatomic Structure for Image-Guided Surgery”), which is incorporated by reference herein in its entirety, discloses example systems.

Medical system 500 may further include operations and support systems (not shown) such as illumination systems, steering control systems, irrigation systems, and/or suction systems. In some embodiments, the medical system 500 may include more than one manipulator assembly and/or more than one master assembly. The exact number of manipulator assemblies may depend on the medical procedure and space constraints within the procedural room, among other factors. Multiple master assemblies may be co-located or they may be positioned in separate locations. Multiple master assemblies may allow more than one operator to control one or more manipulator assemblies in various combinations.

FIG. 5A is a simplified diagram of a medical instrument system 600 according to some embodiments. The medical instrument system 600 includes a flexible elongate device 602 (also referred to as elongate device 602), a drive unit 604, and a medical tool 626 that collectively is an example of a medical instrument 504 of a medical system 500. The medical system 500 may be a teleoperated system, a non-teleoperated system, or a hybrid teleoperated and non-teleoperated system, as explained with reference to FIG. 4. A visualization system 631, tracking system 630, and navigation system 632 are also shown in FIG. 5A and are example components of the control system 512 of the medical system 500. In some examples, the medical instrument system 600 may be used for non-teleoperational exploratory procedures or in procedures involving traditional manually operated medical instruments, such as endoscopy. The medical instrument system 600 may be used to gather (e.g., measure) a set of data points corresponding to locations within anatomic passageways of a patient, such as patient P.

The elongate device 602 is coupled to the drive unit 604. The elongate device 602 includes a channel 621 through which the medical tool 626 may be inserted. The elongate device 602 navigates within patient anatomy to deliver the medical tool 626 to a procedural site. The elongate device 602 includes a flexible body 616 having a proximal end 617 and a distal end 618. In some examples, the flexible body 616 may have an approximately 3 mm outer diameter. Other flexible body outer diameters may be larger or smaller.

Medical instrument system 600 may include the tracking system 630 for determining the position, orientation, speed, velocity, pose, and/or shape of the flexible body 616 at the distal end 618 and/or of one or more segments 624 along flexible body 616, as will be described in further detail below. The tracking system 630 may include one or more sensors and/or imaging devices. The flexible body 616, such as the length between the distal end 618 and the proximal end 617, may include multiple segments 624. The tracking system 630 may be implemented using hardware, firmware, software, or a combination thereof. In some examples, the tracking system 630 is part of control system 512 shown in FIG. 4.

Tracking system 630 may track the distal end 618 and/or one or more of the segments 624 of the flexible body 616 using a shape sensor 622. The shape sensor 622 may include an optical fiber aligned with the flexible body 616 (e.g., provided within an interior channel of the flexibly body 616 or mounted externally along the flexible body 616). In some examples, the optical fiber may have a diameter of approximately 200 μm. In other examples, the diameter may be larger or smaller. The optical fiber of the shape sensor 622 may form a fiber optic bend sensor for determining the shape of flexible body 616. Optical fibers including Fiber Bragg Gratings (FBGs) may be used to provide strain measurements in structures in one or more dimensions. Various systems and methods for monitoring the shape and relative position of an optical fiber in three dimensions, which may be applicable in some embodiments, are described in U.S. Patent Application Publication No. 2006/0013523 (filed Jul. 13, 2005 and titled “Fiber optic position and shape sensing device and method relating thereto”); U.S. Pat. No. 7,772,541 (filed on Mar. 12, 2008 and titled “Fiber Optic Position and/or Shape Sensing Based on Rayleigh Scatter”); and U.S. Pat. No. 8,773,650 (filed on Sep. 2, 2010 and titled “Optical Position and/or Shape Sensing”), which are all incorporated by reference herein in their entireties. Sensors in some embodiments may employ other suitable strain sensing techniques, such as Rayleigh scattering, Raman scattering, Brillouin scattering, and Fluorescence scattering.

In some examples, the shape of the flexible body 616 may be determined using other techniques. For example, a history of the position and/or pose of the distal end 618 of the flexible body 616 can be used to reconstruct the shape of flexible body 616 over an interval of time (e.g., as the flexible body 616 is advanced or retracted within a patient anatomy). In some examples, the tracking system 630 may alternatively and/or additionally track the distal end 618 of the flexible body 616 using a position sensor system 620. Position sensor system 620 may be a component of an EM sensor system with the position sensor system 620 including one or more position sensors. Although the position sensor system 620 is shown as being near the distal end 618 of the flexible body 616 to track the distal end 618, the number and location of the position sensors of the position sensor system 620 may vary to track different regions along the flexible body 616. In one example, the position sensors include conductive coils that may be subjected to an externally generated electromagnetic field. Each coil of position sensor system 620 may produce an induced electrical signal having characteristics that depend on the position and orientation of the coil relative to the externally generated electromagnetic field. The position sensor system 620 may measure one or more position coordinates and/or one or more orientation angles associated with one or more portions of flexible body 616. In some examples, the position sensor system 620 may be configured and positioned to measure six degrees of freedom, e.g., three position coordinates X, Y, Z and three orientation angles indicating pitch, yaw, and roll of a base point. In some examples, the position sensor system 620 may be configured and positioned to measure five degrees of freedom, e.g., three position coordinates X, Y, Z and two orientation angles indicating pitch and yaw of a base point. Further description of a position sensor system, which may be applicable in some embodiments, is provided in U.S. Pat. No. 6,380,732 (filed Aug. 11, 1999 and titled “Six-Degree of Freedom Tracking System Having a Passive Transponder on the Object Being Tracked”), which is incorporated by reference herein in its entirety.

In some embodiments, the tracking system 630 may alternately and/or additionally rely on a collection of pose, position, and/or orientation data stored for a point of an elongate device 602 and/or medical tool 626 captured during one or more cycles of alternating motion, such as breathing. This stored data may be used to develop shape information about the flexible body 616. In some examples, a series of position sensors (not shown), such as EM sensors like the sensors in position sensor 620 or some other type of position sensors may be positioned along the flexible body 616 and used for shape sensing. In some examples, a history of data from one or more of these position sensors taken during a procedure may be used to represent the shape of elongate device 602, particularly if an anatomic passageway is generally static.

FIG. 5B is a simplified diagram of the medical tool 626 within the elongate device 602 according to some embodiments. The flexible body 616 of the elongate device 602 may include the channel 621 sized and shaped to receive the medical tool 626. In some embodiments, the medical tool 626 may be used for procedures such as diagnostics, imaging, surgery, biopsy, ablation, illumination, irrigation, suction, electroporation, etc. Medical tool 626 can be deployed through channel 621 of flexible body 616 and operated at a procedural site within the anatomy. Medical tool 626 may be, for example, an image capture probe, a biopsy tool (e.g., a needle, grasper, brush, etc.), an ablation probe (e.g., a laser ablation tool, radio frequency (RF) ablation tool, cryoablation tool, thermal ablation tool, heated liquid ablation tool, etc.), an electroporation tool, and/or another surgical, diagnostic, or therapeutic tool. In some examples, the medical tool 626 may include an end effector having a single working member such as a scalpel, a blunt blade, an optical fiber, an electrode, and/or the like. Other end types of end effectors may include, for example, forceps, graspers, scissors, staplers, clip appliers, and/or the like. Other end effectors may further include electrically activated end effectors such as electrosurgical electrodes, transducers, sensors, and/or the like.

The medical tool 626 may be a biopsy tool used to remove sample tissue or a sampling of cells from a target anatomic location. In some examples, the biopsy tool is a flexible needle. The biopsy tool may further include a sheath that can surround the flexible needle to protect the needle and interior surface of the channel 621 when the biopsy tool is within the channel 621. The medical tool 626 may be an image capture probe that includes a distal portion with a stereoscopic or monoscopic camera that may be placed at or near the distal end 618 of flexible body 616 for capturing images (e.g., still or video images). The captured images may be processed by the visualization system 631 for display and/or provided to the tracking system 630 to support tracking of the distal end 618 of the flexible body 616 and/or one or more of the segments 624 of the flexible body 616. The image capture probe may include a cable for transmitting the captured image data that is coupled to an imaging device at the distal portion of the image capture probe. In some examples, the image capture probe may include a fiber-optic bundle, such as a fiberscope, that couples to a more proximal imaging device of the visualization system 631. The image capture probe may be single-spectral or multi-spectral, for example, capturing image data in one or more of the visible, near-infrared, infrared, and/or ultraviolet spectrums. The image capture probe may also include one or more light emitters that provide illumination to facilitate image capture. In some examples, the image capture probe may use ultrasound, x-ray, fluoroscopy, CT, MRI, or other types of imaging technology.

In some examples, the image capture probe is inserted within the flexible body 616 of the elongate device 602 to facilitate visual navigation of the elongate device 602 to a procedural site and then is replaced within the flexible body 616 with another type of medical tool 626 that performs the procedure. In some examples, the image capture probe may be within the flexible body 616 of the elongate device 602 along with another type of medical tool 626 to facilitate simultaneous image capture and tissue intervention, such as within the same channel 621 or in separate channels. A medical tool 626 may be advanced from the opening of the channel 621 to perform the procedure (or some other functionality) and then retracted back into the channel 621 when the procedure is complete. The medical tool 626 may be removed from the proximal end 617 of the flexible body 616 or from another optional instrument port (not shown) along flexible body 616.

In some examples, the elongate device 602 may include integrated imaging capability rather than utilize a removable image capture probe. For example, the imaging device (or fiber-optic bundle) and the light emitters may be located at the distal end 618 of the elongate device 602. The flexible body 615 may include one or more dedicated channels that carry the cable(s) and/or optical fiber(s) between the distal end 618 and the visualization system 631. Here, the medical instrument system 600 can perform simultaneous imaging and tool operations.

In some examples, the medical tool 626 is capable of controllable articulation. The medical tool 626 may house cables (which may also be referred to as pull wires), linkages, or other actuation controls (not shown) that extend between its proximal and distal ends to controllably bend the distal end of medical tool 626, such as discussed herein for the flexible elongate device 602. The medical tool 626 may be coupled to a drive unit 604 and the manipulator assembly 502. In these examples, the elongate device 602 may be excluded from the medical instrument system 600 or may be a flexible device that does not have controllable articulation. Steerable instruments or tools, applicable in some embodiments, are further described in detail in U.S. Pat. No. 7,316,681 (filed on Oct. 4, 2005 and titled “Articulated Surgical Instrument for Performing Minimally Invasive Surgery with Enhanced Dexterity and Sensitivity”) and U.S. Pat. No. 9,259,274 (filed Sep. 30, 2008 and titled “Passive Preload and Capstan Drive for Surgical Instruments”), which are incorporated by reference herein in their entireties.

The flexible body 616 of the elongate device 602 may also or alternatively house cables, linkages, or other steering controls (not shown) that extend between the drive unit 604 and the distal end 618 to controllably bend the distal end 618 as shown, for example, by broken dashed line depictions 619 of the distal end 618 in FIG. 5A. In some examples, at least four cables are used to provide independent up-down steering to control a pitch of the distal end 618 and left-right steering to control a yaw of the distal end 281. In these examples, the flexible elongate device 602 may be a steerable catheter. Examples of steerable catheters, applicable in some embodiments, are described in detail in PCT Publication WO 2019/018736 (published Jan. 24, 2019 and titled “Flexible Elongate Device Systems and Methods”), which is incorporated by reference herein in its entirety.

In embodiments where the elongate device 602 and/or medical tool 626 are actuated by a teleoperational assembly (e.g., the manipulator assembly 502), the drive unit 604 may include drive inputs that removably couple to and receive power from drive elements, such as actuators, of the teleoperational assembly. In some examples, the elongate device 602 and/or medical tool 626 may include gripping features, manual actuators, or other components for manually controlling the motion of the elongate device 602 and/or medical tool 626. The elongate device 602 may be steerable or, alternatively, the elongate device 602 may be non-steerable with no integrated mechanism for operator control of the bending of distal end 618. In some examples, one or more channels 621 (which may also be referred to as lumens), through which medical tools 626 can be deployed and used at a target anatomical location, may be defined by the interior walls of the flexible body 616 of the elongate device 602.

In some examples, the medical instrument system 600 (e.g., the elongate device 602 or medical tool 626) may include a flexible bronchial instrument, such as a bronchoscope or bronchial catheter, for use in examination, diagnosis, biopsy, and/or treatment of a lung. The medical instrument system 600 may also be suited for navigation and treatment of other tissues, via natural or surgically created connected passageways, in any of a variety of anatomic systems, including the colon, the intestines, the kidneys and kidney calices, the brain, the heart, the circulatory system including vasculature, and/or the like.

The information from the tracking system 630 may be sent to the navigation system 632, where the information may be combined with information from the visualization system 631 and/or pre-operatively obtained models to provide the physician, clinician, surgeon, or other operator with real-time position information. In some examples, the real-time position information may be displayed on the display system 510 for use in the control of the medical instrument system 600. In some examples, the navigation system 632 may utilize the position information as feedback for positioning medical instrument system 600. Various systems for using fiber optic sensors to register and display a surgical instrument with surgical images, applicable in some embodiments, are provided in U.S. Pat. No. 8,900,131 (filed May 13, 2011 and titled “Medical System Providing Dynamic Registration of a Model of an Anatomic Structure for Image-Guided Surgery”), which is incorporated by reference herein in its entirety.

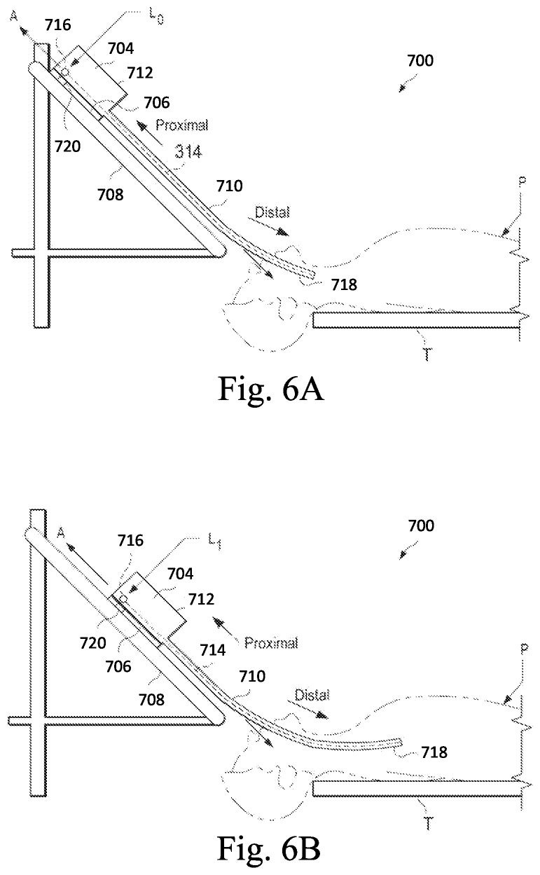

FIGS. 6A and 6B are simplified diagrams of side views of a patient coordinate space including a medical instrument mounted on an insertion assembly according to some embodiments. As shown in FIGS. 6A and 6B, a surgical environment 700 may include a patient P positioned on the patient table T. Patient P may be stationary within the surgical environment 700 in the sense that gross patient movement is limited by sedation, restraint, and/or other means. Cyclic anatomic motion, including respiration and cardiac motion, of patient P may continue. Within surgical environment 700, a medical instrument 704 is used to perform a medical procedure which may include, for example, surgery, biopsy, ablation, illumination, irrigation, suction, or electroporation. The medical instrument 704 may also be used to perform other types of procedures, such as a registration procedure to associate the position, orientation, and/or pose data captured by the sensor system 508 to a desired (e.g., anatomical or system) reference frame. The medical instrument 704 may be, for example, the medical instrument 504. In some examples, the medical instrument 704 may include an elongate device 710 (e.g., a catheter) coupled to an instrument body 712. Elongate device 710 includes one or more channels sized and shaped to receive a medical tool.

Elongate device 710 may also include one or more sensors (e.g., components of the sensor system 508). In some examples, a shape sensor 714 may be fixed at a proximal point 716 on the instrument body 712. The proximal point 716 of the shape sensor 714 may be movable with the instrument body 712, and the location of the proximal point 716 with respect to a desired reference frame may be known (e.g., via a tracking sensor or other tracking device). The shape sensor 714 may measure a shape from the proximal point 716 to another point, such as a distal end 718 of the elongate device 710. The shape sensor 714 may be aligned with the elongate device 710 (e.g., provided within an interior channel or mounted externally). In some examples, the shape sensor 714 may optical fibers used to generate shape information for the elongate device 710.

In some examples, position sensors (e.g., EM sensors) may be incorporated into the medical instrument 704. A series of position sensors may be positioned along the flexible elongate device 710 and used for shape sensing. Position sensors may be used alternatively to the shape sensor 714 or with the shape sensor 714, such as to improve the accuracy of shape sensing or to verify shape information.

Elongate device 710 may house cables, linkages, or other steering controls that extend between the instrument body 712 and the distal end 718 to controllably bend the distal end 718. In some examples, at least four cables are used to provide independent up-down steering to control a pitch of distal end 718 and left-right steering to control a yaw of distal end 718. The instrument body 712 may include drive inputs that removably couple to and receive power from drive elements, such as actuators, of a manipulator assembly.

The instrument body 712 may be coupled to an instrument carriage 706. The instrument carriage 706 may be mounted to an insertion stage 708 that is fixed within the surgical environment 700. Alternatively, the insertion stage 708 may be movable but have a known location (e.g., via a tracking sensor or other tracking device) within surgical environment 700. Instrument carriage 706 may be a component of a manipulator assembly (e.g., manipulator assembly 502) that couples to the medical instrument 704 to control insertion motion (e.g., motion along an insertion axis A) and/or motion of the distal end 718 of the elongate device 710 in multiple directions, such as yaw, pitch, and/or roll. The instrument carriage 706 or insertion stage 708 may include actuators, such as servomotors, that control motion of instrument carriage 706 along the insertion stage 708.

A sensor device 720, which may be a component of the sensor system 508, may provide information about the position of the instrument body 712 as it moves relative to the insertion stage 708 along the insertion axis A. The sensor device 720 may include one or more resolvers, encoders, potentiometers, and/or other sensors that measure the rotation and/or orientation of the actuators controlling the motion of the instrument carriage 706, thus indicating the motion of the instrument body 712. In some embodiments, the insertion stage 708 has a linear track as shown in FIGS. 6A and 6B. In some embodiments, the insertion stage 708 may have curved track or have a combination of curved and linear track sections.

FIG. 6A shows the instrument body 712 and the instrument carriage 706 in a retracted position along the insertion stage 708. In this retracted position, the proximal point 716 is at a position LO on the insertion axis A. The location of the proximal point 716 may be set to a zero value and/or other reference value to provide a base reference (e.g., corresponding to the origin of a desired reference frame) to describe the position of the instrument carriage 706 along the insertion stage 708. In the retracted position, the distal end 718 of the elongate device 710 may be positioned just inside an entry orifice of patient P. Also in the retracted position, the data captured by the sensor device 720 may be set to a zero value and/or other reference value (e.g., I=0). In FIG. 6B, the instrument body 712 and the instrument carriage 706 have advanced along the linear track of insertion stage 708, and the distal end 718 of the elongate device 710 has advanced into patient P. In this advanced position, the proximal point 716 is at a position LI on the insertion axis A. In some examples, the rotation and/or orientation of the actuators measured by the sensor device 720 indicating movement of the instrument carriage 706 along the insertion stage 708 and/or one or more position sensors associated with instrument carriage 706 and/or the insertion stage 708 may be used to determine the position LI of the proximal point 716 relative to the position LO. In some examples, the position LI may further be used as an indicator of the distance or insertion depth to which the distal end 718 of the elongate device 710 is inserted into the passageway(s) of the anatomy of patient P.

One or more components of the embodiments discussed in this disclosure, such as control system 304, 512, may be implemented in software for execution on one or more processors of a computer system. The software may include code that when executed by the one or more processors, configures the one or more processors to perform various functionalities as discussed herein. The code may be stored in a non-transitory computer readable storage medium (e.g., a memory, magnetic storage, optical storage, solid-state storage, etc.). The computer readable storage medium may be part of a computer readable storage device, such as an electronic circuit, a semiconductor device, a semiconductor memory device, a read only memory (ROM), a flash memory, an erasable programmable read only memory (EPROM); a floppy diskette, a CD-ROM, an optical disk, a hard disk, or other storage device. The code may be downloaded via computer networks such as the Internet, Intranet, etc. for storage on the computer readable storage medium. The code may be executed by any of a wide variety of centralized or distributed data processing architectures. The programmed instructions of the code may be implemented as a number of separate programs or subroutines, or they may be integrated into a number of other aspects of the systems described herein. The components of the computing systems discussed herein may be connected using wired and/or wireless connections. In some examples, the wireless connections may use wireless communication protocols such as Bluetooth, near-field communication (NFC), Infrared Data Association (IrDA), home radio frequency (HomeRF), IEEE 802.11, Digital Enhanced Cordless Telecommunications (DECT), and wireless medical telemetry service (WMTS).

The terms “generally,” “approximately,” and “about” used throughout this Specification are used to describe and account for small fluctuations, such as due to variations in processing. For example, they can refer to less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%.

Various general-purpose computer systems may be used to perform one or more processes, methods, or functionalities described herein. Additionally or alternatively, various specialized computer systems may be used to perform one or more processes, methods, or functionalities described herein. In addition, a variety of programming languages may be used to implement one or more of the processes, methods, or functionalities described herein.

While certain embodiments and examples have been described above and shown in the accompanying drawings, it is to be understood that such embodiments and examples are merely illustrative and are not limited to the specific constructions and arrangements shown and described, since various other alternatives, modifications, and equivalents will be appreciated by those with ordinary skill in the art.

1. An energy delivery system, the energy delivery system comprising:
a generator configured to generate an electrical signal having an operating frequency for an ablation operation; and
an ablation probe coupled to the generator and configured to receive the electrical signal from the generator, the ablation probe including:
a flexible body portion including an inner conductor, an outer conductor, and a dielectric between the inner and outer conductors; and
a radiating portion electrically coupled to the inner conductor, the radiating portion configured to radiate energy received from the electrical signal, a length of the radiating portion being between 0.35 and 0.65 times an operating wavelength, the operating wavelength being dependent on the operating frequency of the electrical signal.

2. The energy delivery system of claim 1, further comprising a processor coupled to the generator, wherein the processor is configured to:
determine the operating frequency based on the length of the radiating portion and a speed of a radiated wave in a tissue of interest for the ablation operation; and
cause the generator to generate the electrical signal at the operating frequency.

3. The energy delivery system of claim 1, where the ablation probe provides a reflectivity that is equal to or less than −10 dB.

4. The energy delivery system of claim 1, wherein the operating wavelength corresponds to a frequency of 2.45 GHz.

5. The energy delivery system of claim 1, wherein the operating wavelength corresponds to a frequency band between 915 MHz and 5 GHz.

6. The energy delivery system of claim 1, wherein the radiating portion is separate from the inner conductor.

7. The energy delivery system of claim 1, wherein radiating portion is an extension of the inner conductor that is not surrounded by the outer conductor.

8. The energy delivery system of claim 7, wherein the flexible body portion and the radiating portion comprises a coaxial cable.

9. The energy delivery system of claim 1, wherein the radiating portion is configured to radiate energy to provide a circular ablation zone.

10. The energy delivery system of claim 1, further comprising:
a fluid cooling system including one or more cooling conduits extending along the flexible body portion and providing inlet flow; and
an outer jacket enclosing the flexible body portion, the radiating portion, and the one or more cooling channels in an interior thereof, the interior configured to be filled with saline from the one or more cooling conduits and providing a conduit for outlet flow.

11. The energy delivery system of claim 1, wherein the ablation probe further comprises a cap structure encompassing the radiating portion.

12. The energy delivery system of claim 11, wherein the ablation probe further comprises a barrier encompassing the flexible body portion, the radiating portion, and the cap structure.

13. The energy delivery system of claim 1, further comprising an impedance matching device electrically coupled between the generator and the ablation probe to adjust reflected power to the generator.

14. A method of operating an energy delivery system, the energy delivery system including an ablation probe coupled to a generator and having a flexible body portion with an inner conductor and a radiating portion electrically coupled to the inner conductor, the method comprising:
navigating the ablation probe to position the radiating portion at a target location within a patient;
generating an electrical signal with the generator having an operating frequency; and
radiating energy received from the electrical signal with the radiating portion to ablate tissue of the patient at the target location, a length of the radiating being between 0.35 and 0.65 times an operating wavelength, the operating wavelength being dependent on the operating frequency of the electrical signal.

15. The method of claim 14, further comprising:
determining the operating frequency based on the length of the radiating portion and a speed of a radiated wave in a tissue of interest for the ablation operation; and
causing the generator to generate the electrical signal at the operating frequency.

16. The method of claim 14, wherein radiating energy received from the electrical signal with the radiating portion comprises providing a reflectivity that is equal to or less than −10 dB.

17. The method of claim 14, wherein generating the electrical signal with the generator comprises generating an electrical signal with the generator having an operating wavelength corresponding to a frequency of 2.45 GHz.

18. The method of claim 14, wherein generating the electrical signal with the generator comprises generating an electrical signal with the generator having an operating wavelength corresponding to a frequency band between 915 MHz and 5 GHz.

19. The method of claim 14, wherein radiating energy received from the electrical signal with the radiating portion to ablate tissue of the patient at the target location comprises radiating energy to provide a circular ablation zone.

20. The method of claim 14, further comprising delivering cooling fluid from one or more cooling channels proximate to the radiating portion at least while radiating energy with the radiating portion.

21. The method of claim 14, further comprising adjusting the reflected power to the generator with an impedance matching device electrically coupled between the generator and the ablation probe.
